SEQUENCE LISTING (8 sequences)

SEQ ID NO: 1 - DNA, 32 bp, Artificial Sequence
  Description of Artificial Sequence PCR primer
actcggatgtaaaatatacaaattctactctt

SEQ ID NO: 2 - DNA, 30 bp, Artificial Sequence
  Description of Artificial Sequence PCR primer
tttttcttcaaaggctggattgatatctac

SEQ ID NO: 3 - DNA, 30 bp, Artificial Sequence
  Description of Artificial Sequence PCR primer
aaaggaaaaagctgtagaagaagttgctga

SEQ ID NO: 4 - DNA, 30 bp, Artificial Sequence
  Description of Artificial Sequence PCR primer
tttttcttgaaaagttggatttatagtagt

SEQ ID NO: 5 - DNA, 984 bp, Campylobacter coli
atgaaaaagttattactatgtttagggttgtcaagcgttttatttggtgcagataacaatgtaaaatttgaaatcactcctactttgaatcacaattattttgaaggtaatttagatatggataatcgctatgcaccagggattagactagggtatcattttgatgatttttggcttgatcaattagaactaggtttagaacattactcggatgtaaaatatacaaattctactcttaccaccgatattactagaacttatttgagtgctattaaaggcattgatttaggtgagaaattttatttttatggtttagctggtgggggatatgaggatttttctaaaggcgcttttgataataaaagtggaggatttggccattatggagcaggtttaaaatttcgccttagtgattctttagctttaagacttgaaacaagagatcaaatttctttccatgatgcagatcatagttgggtttcaactttgggtattagttttggctttggcgctaagagagaaaaagttgtagccgaacaagtaaaagaagtagctatagaacctcgtgtagctgtacctacacaatcacaatgtcctgcagagccaagagagggtgctatgctagatgaaaatggttgtgaaaaaacaatttcttttgaaggacattttggttttgataaggtagatatcaatccagcctttgaagaaaaaatcaaagaaattgctcaacttttagatgaaaatgcaagatatgatactattttagagggtcatactgataatataggctcaagagcatacaatcaaaaactttcagaaagacgggctgaaagcgttgcaaaagaacttgaaaaatttggtgtagataaagatcgtatccagacagttggttatggtcaagataaacctcgctcaagaaatgagaccaaagagggtagagcagataacagaagagtggatgctaaatttatcctaagataatga

SEQ ID NO: 6 - DNA, 506 bp, Campylobacter coli
actcggatgtaaaatatacaaattctactcttaccaccgatattactagaacttatttgagtgctattaaaggcattgatttaggtgagaaattttatttttatggtttagctggtgggggatatgaggatttttctaaaggcgcttttgataataaaagtggaggatttggccattatggagcaggtttaaaatttcgccttagtgattctttagctttaagacttgaaacaagagatcaaatttctttccatgatgcagatcatagttgggtttcaactttgggtattagttttggctttggcgctaagagagaaaaagttgtagccgaacaagtaaaagaagtagctatagaacctcgtgtagctgtacctacacaatcacaatgtcctgcagagccaagagagggtgctatgctagatgaaaatggttgtgaaaaaacaatttcttttgaaggacattttggttttgataaggtagatatcaatccagcctttgaagaaaaa

SEQ ID NO: 7 - DNA, 861 bp, Campylobacter jejuni
gcaagtgttttatttggtcgtgataacaatgtaaaatttgaaatcactccaactttaaactataattactttgaaggtaatttagatatggataatcgttatgcaccagggattagacttggttatcattttgacgatttttggcttgatcaattagaatttgggttagagcattattctgatgttaaatatacaaatactaataaaactacagatattacaagaacttatttgagtgctattaaaggtattgatgtaggtgagaaattttatttctatggtttagcaggtggaggatatgaggatttttcaaatgctgcttatgataataaaagcggtggatttggacattatggcgcgggtgtaaaattccgtcttagtgattctttggctttaagacttgaaactagagatcaaattaattttaatcatgcaaaccataattgggtttcaactttaggtattagttttggttttggtggcaaaaaggaaaaagctgtagaagaagttgctgatactcgtccagctccacaagcaaaatgtcctgttccttcaagagaaggtgctttgttagatgaaaatggttgcgaaaaaactatttctttggaaggtcattttggttttgataaaactactataaatccaacttttcaagaaaaaatcaaagaaattgcaaaagttttagatgaaaatgaaagatatgatactattcttgaaggacatacagataatatcggttcaagagcttataatcaaaagctttctgaaagacgtgctaaaagtgttgctaatgaacttgaaaaatatggtgtagaaaaaagtcgcatcaaaacagtaggttatggtcaagataatcctcgc

SEQ ID NO: 8 - DNA, 175 bp, Campylobacter jejuni
aaaggaaaaagctgtagaagaagttgctgatactcgtccagctccacaagcaaaatgtcctgttccttcaagagaaggtgctttgttagatgaaaatggttgcgaaaaaactatttctttggaaggtcattttggttttgataaaactactataaatccaacttttcaagaaaaa

(19) United States
(12) Patent Application Publication
(10) Pub. No.: US 2003/0113757 A1
(43) Pub. Date: Jun. 19, 2003

(54) Rapid and specific detection of campylobacter

(76) Inventor: John W. Czajka, West Grove, PA (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/216,338

(22) Filed: Aug. 8, 2002

Related U.S. Application Data
(60) Provisional application No. 60/310,882, filed on Aug. 8, 2001.

(51) Int. Cl. C12Q 1/68; C12P 19/34

(52) U.S. Cl. 435/6; 435/91.2

(57) ABSTRACT

The present invention provides a method for specifically detecting pathogenic Campylobacter species in a complex sample. The target pathogenic Campylobacter species can be Campylobacter jejuni or Campylobacter coli. The complex sample can be a food sample, water sample, or selectively enriched food matrix. The method of detection utilizes PCR amplification with, or without, an internal positive control, and appropriate primer pairs. Multiple species can be detected in the same reaction. The reagents necessary to perform the method can be supplied as a kit and/or in tablet form.

This application claims the priority benefit of U.S. Provisional Application 60/310,882 filed Aug. 8, 2001, the disclosure of which is hereby incorporated by reference in its entirety.

### FIELD OF THE INVENTION

This invention relates to a rapid method for detection of Campylobacter bacteria, oligonucleotide molecules and reagents kits useful therefor. Specifically the target bacteria are detected with PCR in a homogeneous or gel-based format by means of labeling DNA amplification products with a fluorescent dye.

### BACKGROUND OF THE INVENTION

Campylobacter species are the most common bacteria associated with foodborne gastroenteritis worldwide. The vast majority (in some areas, approximately 90%) of cases are associated with Campylobacter jejuni, and the remaining cases are caused by C. coli, although a minority of cases are associated with other species such as C. upsaliensis and C. lari.

The organisms often persist in healthy animals such as cattle and poultry, which can serve as reservoirs for human disease. Identification of Campylobacter isolates is often difficult and the differentiation between C. jejuni and C. coli relies on one phenotypic test-the hydrolysis of hippurate. Misidentification of species can create difficulties in surveillance monitoring, epidemiology, and detection. As a consequence, the source of most infections is often unknown.

Although there is a need to be able to detect and differentiate species of Campylobacter, currently available techniques have many drawbacks. In particular, there is no satisfactory detection method that is sensitive, convenient and capable of differentiating the two main pathogenic species.

Conventional enrichment culture techniques lack sensitivity and are time-consuming. For example, it is known that C. jejuni does not grow in foodstuffs and its numbers are low compared to the high background of indigenous microflora. Also, surface viable counts of Campylobacter can decrease rapidly as potentially culturable cells are often lost during sample preparation, storage and transportation. C. jejuni is known to enter a non-culturable, yet viable and infective form, when subjected to environmental stresses, such as pH or temperature extremes, increased oxygen level or nutrient depletion. Furthermore, culture enrichment media often contain antibiotics that may inhibit Campylobacter growth.

A number of recombinant DNA-based detection methods, particularly DNA amplification-based methods, are also known in the art. However, those methods either do not discriminate between C. coli and C. jejuni (see e.g. Giesendorf et al. 1992, Appl. Environ. Microbiol., 58:3804-3808 and Wegmuller et al., 1993, Appl. Environ. Microbiol., vol. 59:2161-2165), or requires an additional restriction digesting step to differentiate between the species (e.g., Fox et al. U.S. Pat. No. 6,080,547), or otherwise requires the combination of a restriction enzyme and probe for species identification (e.g., the Strand Displacement Amplification method disclosed in McMillian et al., U.S. Pat. No. 6,066,461, which uses a radioactive isotope for probe-based detection of the SDA product). In addition, the methods of Fox et al. and McMillan et al, have poor sensitivities (approximately 100 cells/reaction), which may not be satisfactory for use within the food, water, and clinical fields.

Lawson et al, 1999, J. Clin. Microbiol. 37:3860-3864, discloses a method that uses a complex combination of PCR assays and probe detection to achieve the detection and identification of C. coli and C. jejuni. A first PCR was used to amplify the DNA from C. coli, C. jejuni, C. upsaliensis, C. lari, and C. helveticus, followed by a probe hybridization to determine if the isolate is C. coli/jejuni, C. upsaliensis, C. lari, or C. helveticus. In order to differentiate C. coli from C. jejuni, they then perform a second PCR that has four primers. The second PCR that was used to differentiate C. coli from C. jejuni was unable to speciate 35 out of 478 isolates that were C. coli/jejuni positive for the probe identification.

Gonzalez et al., 1997, J. Clin. Microbiol. 35:759-763, discloses a PCR-based method for the detection and identification of C. coli and C. jejuni based on the ceuE gene. The detection of both species requires two different PCR reactions, one for C. coli and one for C. jejuni. The primer sets showed 100% inclusivity and 100% exclusivity on the limited number of strains tested (12 C. jejuni and 16 C. coli). This test, however, produces amplicons that are 894 bp for C. coli and 897 bp for C. jejuni, and are not distinguishable by agarose gel electrophoresis. Therefore, this test does not allow identification of one species in the presence of the other in the same reaction tube.

Multiplex PCR, or multiplexing, is the art of combining multiple primer sets in one PCR, thus allowing for the identification of more than one target. None of the primer sets previously described in the art could be multiplexed due to different optimal reaction temperatures or identical amplicon size for both primer sets of interest.

There is a need, therefore, for a PCR-based method and suitable PCR primers that can achieve (1) one-step species identification for both C. coli and C. jejune, even when both species are present in the sample, without interference from each other, (2) a test that allows for multiplexing, (3) a test with a sensitivity between one and ten cells per reaction and (4) a test that has the ability to quantify the number C. coli and/or C. jejuni present.

### SUMMARY OF THE INVENTION

The present invention provides a method for detecting a pathogenic Campylobacter species, in a sample, comprising: (i) preparing the sample for PCR amplification; (ii) performing PCR amplification of the sample using a combination of PS1 and PS2 primers; and (iii) examining the PCR amplification result, whereby a positive amplification indicates the presence of a pathogenic Campylobacter species.

The detection methods of the present invention further encompass steps comprising at least one of the following processes: (i) bacterial enrichment; (ii) separation of bacterial cells from the sample; (iii) cell lysis; and (iv) total DNA extraction.

In another embodiment of the invention the target pathogenic Campylobacter species can be Campylobacter jejuni or Campylobacter coli.

In still another embodiment the sample comprises a food sample, water sample, or selectively enriched food matrix.

The present invention further encompasses the use of polynucleotide primers for the specific detection of Campylobacter jejuni or Campylobacter coli consisting essentially of the nucleic acid sequences such as, but not limited to, SEQ ID NOs: 1-4.

A further embodiment of the present invention involves a kit for the detection of a pathogenic Campylobacter species, the kit comprising: (i) at least one pair of PCR primers selected from the group consisting of PS1 and PS2; and (ii) a mixture of suitable PCR reagents comprising a thermostable DNA polymerase.

In yet another embodiment the mixture of suitable PCR reagents is provided in a tablet.

### SUMMARY OF THE SEQUENCES

SEQ ID NO: 1 is the nucleotide sequence of a 5′ primer to a region of the cadF gene that will specifically detect Campylobacter coli in a polymerase chain reaction with bacterial DNA and SEQ ID NO: 2.

SEQ ID NO: 2 is the nucleotide sequence of a 3′ primer to a region of the cadF gene that will specifically detect Campylobacter coli in a polymerase chain reaction with bacterial DNA and SEQ ID NO: 1.

SEQ ID NO: 3 is the nucleotide sequence of a 5′ primer to a region of the cadF gene that will specifically detect Campylobacter jejuni in a polymerase chain reaction with bacterial DNA and SEQ ID NO: 4.

SEQ ID NO: 4 is the nucleotide sequence of a 3′ primer to a region of the cadF gene that will specifically detect Campylobacter jejuni in a polymerase chain reaction with bacterial DNA and SEQ ID NO: 3.

SEQ ID NO: 5 is the nucleotide sequence of the cadF gene from Campylobacter coli.

SEQ ID NO: 6 is the nucleotide sequence representing one strand of the PCR amplification product of the primers in SEQ ID NOs: 1 and 2.

SEQ ID NO: 7 is the nucleotide sequence of the cadF gene from Campylobacter jejuni.

SEQ ID NO: 8 is the nucleotide sequence representing one strand of the PCR amplification product of the primers in SEQ ID NOs: 3 and 4.

### BRIEF DESCRIPTION OF THE DRAWINGS

FIG. 1 shows the process of melting curve analysis. The change in fluorescence of the target DNA is captured during melting. Mathematical analysis of the negative log of fluorescence divided by the change in temperature plotted against the change in temperature results in the graphical peak known as a melting curve.

FIG. 2 is a gel photograph showing C. coli and C. jejuni results. Leftmost lane, top and bottom, DNA mass ladder. Lanes 2-9, top and bottom, individual sample results, with a positive C. jejuni band running at 175 bp (lanes 2-6 and 8-9) and a C. coli band at 506 bp (lane 7).

FIG. 3 shows a C. coli melting curve. The temperature peaks at 82.5° C. indicating the presence of C. coli.

FIG. 4 shows a C. jejuni/C. coli melting curve. The temperature peaks at 82.5° C. for C. coli but at 80.5° C. for C. jejuni, making it possible to detect both organisms in the same reaction.

FIG. 5 shows an internal positive control melting curve for C. coli. The temperature peaks at 82.5° C. for C. coli but at 78° C. for the internal positive control (INPC), so that the target amplicon and the INPC can be monitored simultaneously. The INPC controls for the fidelity of the PCR reaction in the sample solution even when the target amplicon is not present, thereby increasing the efficiency of system throughput.

### DETAILED DESCRIPTION OF THE INVENTION

The present invention provides a method to detect, identify, and differentiate pathogenic Campylobacter species, i.e. C. jejuni and C. coli based on the amplification of, or hybridization to, a part of the cadF gene of the bacteria.

Nucleic acid regions that are unique to either Campylobacter jejuni or Campylobacter coli have been identified within the cadF gene. Oligonucleotide primers suitable for the polymerase chain reaction (PCR) amplification have been developed for the detection and identification of either of the above mentioned species. These oligonucleotide primers would also be useful for other nucleic acid amplification methods such as the ligase chain reaction (LCR) (Backman et al., 1989, EP 0 320 308; Carrino et al., 1995, J. Microbiol. Methods 23: 3-20); nucleic acid sequence-based amplification (NASBA), (Carrino et al., 1995, supra); and self-sustained sequence replication (3SR) and ‘Q replicase amplification’ (Pfeffer et al., 1995 Veterinary Res. Comm., 19: 375-407).

The oligonucleotides of the instant invention are also used as hybridization probes. Hybridization using DNA probes have been frequently used for the detection of pathogens in food, clinical and environmental samples, and the methodology are generally known to a skilled in the art. It is generally recognized that the degree of sensitivity and specificity of probe hybridization is lower than that achieved through the previously described amplification techniques.

Both amplification-based and hybridization-based methods using the oligonucleotides of the invention may be used to confirm the identification of C. jejuni and C. coli in enriched or even purified culture. A preferred embodiment of the instant invention comprises (1) culturing a complex sample mixture in a non-selective growth media to resuscitate the target bacteria, (2) releasing total target bacterial DNA and (3) subjecting the total DNA to amplification protocol with a primer pair of the invention

More importantly, however, the oligonucleotides may be used to detect and identify the two species directly in complex samples such as clinical specimens from humans or animals, or from samples of contaminated food or water, without the need for pre-enrichment or purification.

As will be explained in more detail below, the amplified nucleic acids are identified by, for example, gel electrophoresis, nucleic acid probe hybridization, fluorescent end point measurement, and melting curve analysis.

This invention allows for the rapid and accurate determination of whether a sample contains C. jejuni, or C. coli, or both.

Primers/Oligonucleotides: Design and Sequence Information

The oligonucleotides of the instant invention were designed in order to identify specifically Campylobacter coli or Campylobacter jejuni from a complex mixture without giving false positives due to the presence of other Campylobacter species or other bacteria. The oligonucleotides may also be used to amplify either of the two Campylobacter species. Multiple primers and combinations were tested under a variety of reaction conditions. Two primer sets PS1, (specific for C. coli, and consisting of two primers having the sequences of SEQ ID NO: 1 and SEQ ID NO: 2,), and PS2 (specific for C. jejuni, consisting of two primers having the sequence of SEQ ID NO: 3 and SEQ ID NO: 4) were designed using the cadF gene sequence (Konkel et al., 1999, J. Clin. Microbiol. 37: 510-517).

Both primer sets demonstrated that they can amplify 100% of their intended target bacterial isolates, and none of the numerous non-target bacterial isolates.

The PCR amplification products for Campylobacter coli and Campylobacter jejuni are shown in SEQ ID NOs: 6 and 8, respectively. A primer design program (Oligo5.0, National Biosciences Inc., Plymouth, Minn.) was used that eliminates detrimental primer configurations such as primer dimers or hairpins, while maintaining specificity for each target organism.

Sample Preparation

The oligonucleotides and methods according to the instant invention may be used directly with any suitable clinical or environmental samples, without any need for sample preparation. In order to achieve higher sensitivity, and in situations where time is not a limiting factor, it is preferred that the samples be pre-treated, and pre-amplification enrichment is performed.

The minimum industry standard for the detection of food-borne bacterial pathogens is a method that will reliably detect the presence of one pathogen cell in 25 g of food matrix as described in Andrews et al., 1984, “Food Sample and Preparation of Sample Homogenate”, Chapter 1 in Bacteriological Analytical Manual, 8th Edition, Revision A, Association of Official Analytical Chemists, Arlington, Va. In order to satisfy this stringent criterion, enrichment methods and media have been developed to enhance the growth of the target pathogen cell in order to facilitate its detection by biochemical, immunological or nucleic acid hybridization means. Typical enrichment procedures employ media that will enhance the growth and health of the target bacteria and also inhibit the growth of any background or non-target microorganisms present. For example the U.S. Food and Drug Administration (FDA) endorses a Campylobacter assay procedure described in Hunt et al., 1995, “Isolation and Identification of Campylobacter Species in Food and Water,” Chapter 7 in Bacteriological Analytical Manual, 8th Edition, Association of Official Analytical Chemists, Arlington, Va. In this procedure, the selective broth medium Bolton's broth is used to restore injured Campylobacter cells to a stable condition and to promote growth. Selective media have been developed for a variety of bacterial pathogens and one of skill in the art will know to select a medium appropriate for the particular organism to be enriched. A general discussion and recipes of non-selective media are described in the FDA Bacteriological Analytical Manual. (1998) published and distributed by the Association of Analytical Chemists, Suite 400, 2200 Wilson Blvd, Arlington, Va. 22201-3301.

After selective growth, a sample of the complex mixtures is removed for further analysis. This sampling procedure may be accomplished by a variety of means well known to those skilled in the art. In a preferred embodiment, 5 ul of the enrichment culture is removed and added to 200 ul of lysis solution containing protease. The lysis solution is heated at 37° C. for 20 min followed by protease inactivation at 95° C. for 10 min as described in the BAX® systems User's Guide, Qualicon, Inc., Wilmington, Del.

Amplification Conditions

A skilled person will understand that any generally acceptable PCR conditions may be used for successfully detecting the target Campylobacter bacteria using the oligonucleotides of the instant invention, and depending on the sample to be tested and other laboratory conditions, routine optimization for the PCR conditions may be necessary to achieve optimal sensitivity and specificity. Optimally, they achieve PCR amplification products from all of the intended specific targets while giving no PCR product for other, non-target species.

In a preferred embodiment, the following cycling conditions were used. Forty-five microliters of lysate was added to a PCR tube containing one BAX® reagent tablet (manufactured by Qualicon, Inc., Wilmington, Del.), the tablet containing Taq DNA polymerase, deoxynucleotides, SYBR® Green (Molecular Probes, Eugene, Oreg.), and buffer components, and 5 microliters of primer mix to achieve a final concentration in the PCR of 0.150 micromoles for each primer. PCR cycling conditions were as follows: 94° C. for two minutes, 38 cycles of 94° C. for 15 seconds, 65° C. for two minutes, and 72° C. for one minute.

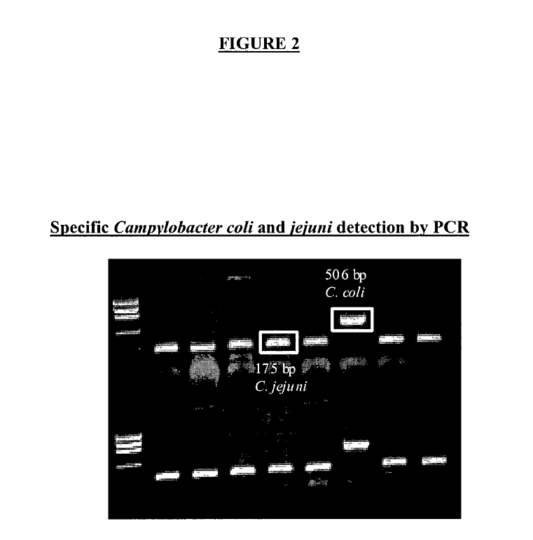

The PCR reaction was then subjected to electrophoresis on an ethidium bromide-stained 2% agarose gel, run for 30 min at 200 V. The results were then visualized under UV light (FIG. 2).

Homogenous PCR

Homogenous PCR refers to a method for the detection of DNA amplification products where no separation (such as by gel electrophoresis) of amplification products from template or primers is necessary. Homogeneous detection of the present invention is typically accomplished by measuring the level of fluorescence of the reaction mixture in the presence of a fluorescent dye.

In a preferred embodiment, DNA melting curve analysis is used, particularly with the BAX® System hardware and reagent tablets from Qualicon InC. (Wilmington, Del.). The details of the system are given in PCT Publication Nos. WO 97/11197 and WO 00/66777, the contents of which are hereby incorporated by reference.

Melting Curve Analysis

Melting curve analysis detects and quantifies double stranded nucleic acid molecule (“dsDNA” or “target”) by monitoring the fluorescence of the amplified target (“target amplicon”) during each amplification cycle at selected time points.

As is well known to the skilled artisan, the two strands of a dsDNA separate or melt, when the temperature is higher than its melting temperature. Melting of a dsDNA molecule is a process, and under a given solution condition, melting starts at a temperature (designated T_{MS} hereinafter), and completes at another temperature (designated T_{ME} hereinafter). The familiar term, Tm, designates the temperature at which melting is 50% complete.

A typical PCR cycle involves a denaturing phase where the target dsDNA is melted, a primer annealing phase where the temperature optimal for the primers to bind to the now-single-stranded target, and a chain elongation phase (at a temperature T_{E}) where the temperature is optimal for DNA polymerase to function. According to the present invention, T_{MS} should be higher than T_{E}, and T_{ME}should be lower (often substantially lower) than the temperature at which the DNA polymerase is heat-inactivated. Melting characteristics are effected by the intrinsic properties of a given dsDNA molecule, such as deoxynucleotide composition and the length of the dsDNA.

Intercalating dyes will bind to doublestranded DNA. The dye/dsDNA complex will fluoresce when exposed to the appropriate excitation wavelength of light, which is dye dependent and the intensity of the fluorescence may be proportionate to concentration of the dsDNA. Methods taking advantage of the use of DNA intercalating dyes to detect and quantify dsDNA are known in the art. Many dyes are known and used in the art for these purposes. The instant methods also take advantage of such relationship. An example of such dyes includes intercalating dyes. Examples of such dyes include, but are not limited to, SYBR Green-I®, ethidium bromide, propidium iodide, TOTO®-1 {Quinolinium, 1-1′-[1 ,3-propanediylbis[(dimethyliminio)-3,1-propanediyl]]bis[4-[(3-methyl-2(3H)-benzothiazolylidene)methyl]]-, tetraiodide}, and YoPro®{Quinolinium, 4-[(3-methyl-2(3H)-benzoxazolylidene)methyl]-1-[3-(trimethylammonio)propyl]-,diiodide}. Most preferred dye for the instant invention is a non-asymmetrical cyanide dye such as SYBR Green-I®, manufactured by Molecular Probes, Inc. (Eugene, Oreg.).

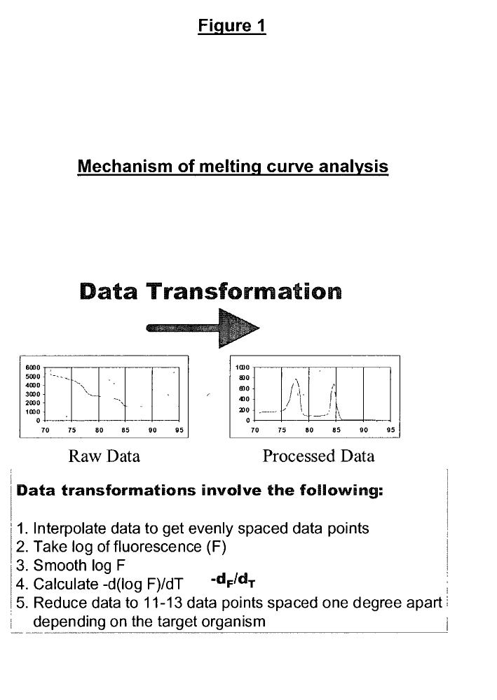

Melting curve analysis is achieved by monitoring the change in fluorescence while the temperature is increased. When the temperature reaches the T_{MS} specific for the PCR amplicon, the dsDNA begins to denature. When the dsDNA denatures, the intercalating dye dissociates from the DNA and fluorescence decreases. Mathematical analysis of the negative log of fluoresces divided by the change in temperature plotted against the change in temperature results in the graphical peak known as a melting curve (FIG. 1).

The data transformation process shown in FIG. 1 involve the following:

1. Interpolate data to get evenly spaced data points

2. Take a log of the fluorescence (F)

3. Smooth log F

4. Calculate-d(log F)/dT

5. Reduce data to 11-13 data points spaced one degree apart (depending on the target organism).

The instant detection method can be used to detect and quantify target dsDNAs, from which the presence and level of target organisms can be determined. The instant method is very specific and sensitive. The fewest number of target dsDNA detectable is between one and 10.

Internal Positive Control

In a preferred embodiment the PCR tablet for pathogenic organisms contains an internal positive control. The advantages of an internal positive control contained within the PCR reaction have been previously described (PCT Application No. WO 97/11197 published on Mar. 27, 1997, the contents of which are hereby incorporated by reference) and include (i) the control may be amplified using a single primer; (ii) the amount of the control amplification product is independent of any target DNA contained in the sample; (iii) the control DNA can be tabletted with other amplification reagents for ease of use and high degree of reproducibility in both manual and automated test procedures; (iv) the control can be used with homogeneous detection, i.e., without separation of product DNA from reactants and (v) the internal control has a melting profile that is distinct from other potentially produced amplicons in the reaction. Control DNA will be of appropriate size and base composition to permit amplification in a primer directed amplification reaction. The control DNA sequence may be obtained from the target bacteria, or from another source, but must be reproducibly amplified under the same conditions that permit the amplification of the target amplicon DNA. The control reaction is useful to validate the amplification reaction. Amplification of the control DNA occurs within the same reaction tube as the sample that is being tested, and therefore indicates a successful amplification reaction when samples are target negative, i.e. no target amplicon is produced. In order to achieve significant validation of the amplification reaction a suitable number of copies of the control DNA must be included in each amplification reaction.

According to a preferred embodiment, an automated thermal cycler with fluorescence detection capabilities such as the Perkin-Elmer 7700 Sequence Detection System available from the Perkin-Elmer Corporation is used. Fluorescence data are exported and processed with the help of a data processing device such as a personal computer, with various transformations when necessary. Methods and instruments for such automated operation are apparent to a skilled person and are exemplified in the examples that follow.

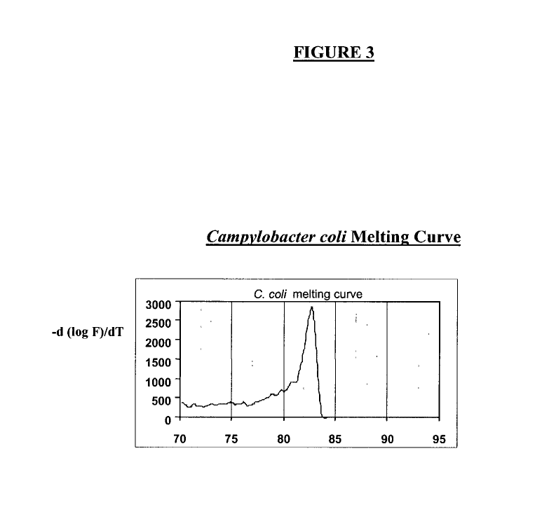

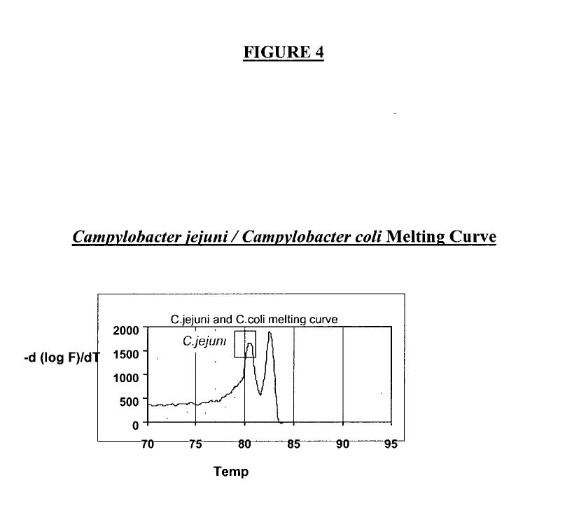

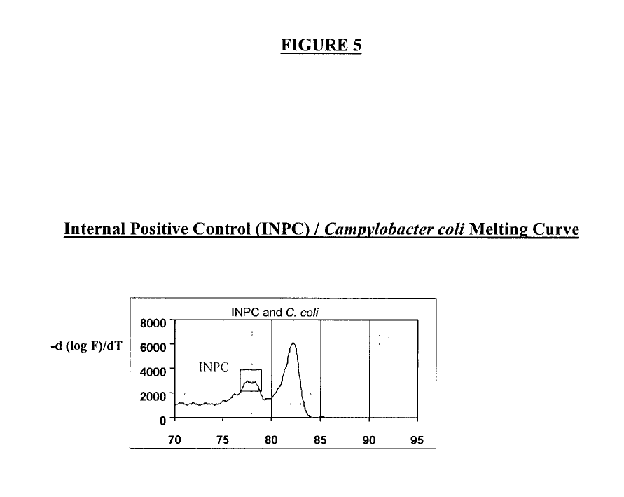

Several of the amplifications were analyzed using the Automated BAX® system and melting curve analysis. FIG. 3 shows the melting curve for a C. coli-positive sample, which has a melting curve peak at 82.5° C. The C. jejuni PCR product melts out at 80.5° C. (data not shown). FIG. 4 shows the melting curve analysis for a sample that contained both C. coli and C. jejuni. FIG. 5 shows the melting curve analysis for a C. coli-positive sample in which the internal positive control was added to the Campylobacter multiplex PCR. The internal positive control melts out at 78° C., which is clearly distinguishable from the Campylobacter amplicons.

Multiplex PCR

The method according to the instant invention can also be used to detect simultaneously multiple target amplicons (“multiplex detection”) . The technique of multiplex PCR provides many benefits over the conventional “one target” PCR. Multiplex PCR requires the development of PCR primers for multiple targets that are specific for their individual target and compatible with each other. In order for multiplex primers to be compatible, all of the primers must anneal at the same annealing temperature, under the same chemical reaction conditions. Also, the primers must not cross-react or anneal to other multiplex targets that the primer was not specifically designed for, and the primers must not cross-react or bind to the other multiplex primers during the amplification. For agarose gel detection of the PCR, the amplicons need to be distinct in size so that each amplicon migrates through the agarose gel at a different rate, resulting in visibly distinct bands. For homogeneous detection, the target amplicons should have distinct melting curve characteristics, which would allow for the specific identification of each target melting curve peak.

In order to prevent the misidentification of these Campylobacter species, as well as to aid in the monitoring of Campylobacter populations, a multiplex PCR assay has been developed for the detection and species identification of both C. jejuni and C. coli. Sequence analysis of a common bacterial gene was used to develop one primer set for C. jejuni and one for C. coli. These primers were used with a polymerase chain reaction (PCR) protocol that utilized either agarose gel detection or a homogeneous format that combines DNA amplification and detection to determine the presence or absence of a specific target.

Bacterial strains were tested by adding 45 microliters of lysed cells to a PCR tube containing one reagent tablet and all four primers. Reagent tablets contain DNA polymerase, deoxynucleotides, and buffer components. The results for the PCR were determined by agarose gel electrophoresis for each of 256. Testing of the multiplex PCR resulted in 100% inclusivity for the 130 strains of C. jejuni and 66 strains C. coli for each respective primer set. The primers also showed 100% exclusivity when tested on 60 isolates representing five other Campylobacter species and three Arcobacter species. Current work with this multiplex PCR involves the development of a homogeneous detection format based on melting curve analysis and the incorporation of an internal positive control.

Kits and Reagent Tablets

Any suitable nucleic acid replication composition can be used for the instant invention. Typical PCR amplification composition contains for example, dATP, dCTP, dGTP, dTTP, target specific primers and a suitable polymerase. If nucleic acid composition is in liquid form, suitable buffers known in the art are used (Sambrook, J. et al. 1989, Molecular Cloning: A Laboratory Manual, Second Edition, Cold Spring Harbor Laboratory Press).

Alternatively if the composition is contained in a tabletted reagent, then typical tabletting reagents are included such as stabilizers and the like.

Within the context of the present invention replication compositions will be modified depending on whether they are designed to be used to amplify target DNA or the control DNA. Replication compositions that will amplify the target DNA, (test replication compositions) will include (i) a polymerase (generally thermostable), (ii) a primer pair capable of hybridizing to the target DNA and (iii) necessary buffers for the amplification reaction to proceed. Replication compositions that will amplify the control DNA (positive control, or positive replication composition) will include (i) a polymerase (generally thermostable) (ii) the control DNA; (iii) at least one primer capable of hybridizing to the control DNA; and (iv) necessary buffers for the amplification reaction to proceed. In some instances it may be useful to include a negative control replication composition. The negative control composition will contain the same reagents as the test composition but without the polymerase. The primary function of such a control is to monitor spurious background fluorescence in a homogeneous format when the method employs a fluorescent means of detection.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Materials and methods suitable for the maintenance and growth of bacterial cultures are well known in the art. Techniques suitable for use in the following examples may be found in Manual of Methods for Genus Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994) or Thomas D. Brock in Biotechnology: A Textbook of Industrial Microbiology, Second Edition (1989) Sinauer Associates, Inc., Sunderland, Mass. or Bacteriological Analytical Manual. 6th Edition, Association of Official Analytical Chemists, Arlington, Va. (1984).

The selective medium used to grow the Campylobacter strains that were used in the following examples was Bolton broth obtained from Hardy Diagnostics (Santa Maria, Calif.).

All other reagents and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Primers (SEQ ID NOs: 1-4), were prepared by Research Genetics, Huntsville, Ala. The following are reagents that were used in the PCR: Sybr® Green (Molecular Probes, Eugene, Oreg.), Taq DNA Polymerase (Roche Diagnostics, Indianapolis, Ind.), deoxynucleotides (Boehringer Mannheim, Indianapolis, Ind.), buffer (EM Science, Cincinnati, Ohio).

The meaning of abbreviations is as follows: “h” means hour(s), “min” means minute(s), “sec” means second(s), “d” means day(s), “mL” means milliliters.

### Example 1

### Amplification of Campylobacter Specific DNA Fragments

Primer pairs were designed to specifically identify Campylobacter coli or Campylobacter jejuni from a complex mixture without giving false positives to other Campylobacter species or other bacteria. Multiple primers and combinations were tested under a variety of reaction conditions. The optimized primers and reaction conditions are different from those previously described for PCR based detection of Campylobacter. Two primer sets (PS1 specific for Campylobacter coli, and PS2 specific for Campylobacter jejuni, Table 1) were designed using the published cadF gene sequences, SEQ ID NOs: 5 and 7, respectively (Konkel et al. (1999) J. Clin Micro 37: 510-517). The PCR amplification products for Campylobacter coli and Campylobacter jejuni are shown in SEQ ID NOs: 6 and 8, respectively. A primer design program (Oligo5.0, National Biosciences Inc., Plymouth, Minn.) was used that eliminates detrimental primer configurations such as primer dimers or hairpins, while maintaining specificity for each target organism.

TABLE 1

| Primer set | SEQ ID NO | Target |
|---|---|---|
| PS1 | SEQ ID NO:1 and SEQ ID NO:2 | C. coli |
| PS2 | SEQ ID NO:3 and SEQ ID NO:4 | C. jejuni |

The two primer sets were run under various PCR cycling conditions and at various primer concentrations to determine the optimal conditions for the reaction. The desired result gave PCR amplification products for all of the species specific targets while giving no PCR product for other species. The optimal conditions were tested against lysates for two C. coli strains and five C. jejuni strains. The following cycling conditions were tested with the above mentioned primer sets at a concentration of 1.0 μM for each primer: 94° C., 2 min initial DNA denaturation, followed by 38 cycles of 94° C., 30 sec, denaturation 65° C., 2 min primer annealing and 72° C., 1 min for primer elongation. The determination of a positive PCR was achieved with agarose gel electrophoresis as mentioned above. A positive reaction for C. coli resulted in the appearance of a DNA band of 506 bp in size, while a positive for C. jejuni resulted in a DNA band of 175 bp in size (FIG. 2).

TABLE 2
Results from PCR with Primer Set 2 and C. jejuni Samples

| Campylobacter strain | PS1 | PS2 |
|---|---|---|
| C. coli 9676 | + | − |
| C. coli 9697 | + | − |
| C. jejuni 9698 | − | + |
| C. jejuni 9695 | − | + |
| C. jejuni 9694 | − | + |
| C. jejuni 9693 | − | + |

The primer sets PS1 and PS2 were combined in one multiplex PCR and tested against a panel of bacterial strains that consisted of C. coli, C. jejuni, additional Campylobacter species, and non-Campylobacter bacteria. Results shown in Table 3 and 4.

TABLE 3
Non-Campylobacter strains tested. All strains were negative
for both primer sets.

| | # of strains |
|---|---|
| Genus/species | tested |
| Aeromonas salmonicida | 1 |
| Bacillus cereus | 3 |
| B. subtilis | 1 |
| B. thuringiensis | 1 |
| Citrobacter freundii | 2 |
| Enterobacter | 1 |
| agglomerans | |
| E. casseliflavus | 2 |
| E. cecorum | 2 |
| E. cloacae | 6 |
| E. durans | 1 |
| E. faecalis | 3 |
| E. faecium | 3 |
| E. gallinarum | 2 |
| E. hirae | 1 |
| E. malodoratus | 1 |
| E. mundti | 1 |
| E. pseudoavium | 1 |
| E. saccharolyticus | 1 |
| Enterococcus avium | 2 |
| E. faecalis | 4 |
| Klebsiella pneumoniae | 2 |
| Lactococcus garviae | 2 |
| L. lactis | 4 |
| L. plantarum | 1 |
| L. raffinolactis | 1 |
| M. luteus | 1 |
| Leuconostoc | 1 |
| mesenteroides | |
| Listeria ivanovii | 2 |
| L. monocytogenes | 3 |
| Micrococcus kristinae | 1 |
| M. lylae | 1 |
| M. roseus | 1 |
| M. sedentarius | 1 |
| M. varians | 1 |
| Pediococcus acidilactici | 1 |
| P. pentosaceus | 1 |
| Proteus mirabilis | 3 |
| P. species | 1 |
| P. vulgaris | 1 |
| Pseudomonas | 2 |
| aeruginosa | |
| P. fluorescens | 4 |
| P. putida | 1 |
| P. stutzeri | 1 |
| Ralstonia picketii | 1 |
| Rhodococcus egui | 1 |
| Salmonella drypool | 1 |
| S. enteritidis | 9 |
| S. heidelberg | 1 |
| S. infantis | 1 |
| S. pullorum | 10 |
| S. reading | 1 |
| S. saintpaul | 1 |
| S. saphra | 1 |
| S. schwarzengrund | 1 |
| S. species | 5 |
| S. thomasville | 1 |
| S. typhimurium | 13 |
| S. worthington | 4 |
| Serracia marcescens | 2 |
| Shigella sonnei | 4 |
| S. species | 3 |
| Staphylococcus aureus | 1 |
| S. capitis | 5 |
| S. capitis | 1 |
| S. caprae | 2 |
| S. carnosus | 1 |
| S. caseolyticus | 1 |
| S. chromogenes | 4 |
| S. cohnii | 5 |
| S. delphini | 1 |
| S. epidermidis | 6 |
| S. epidermidis | 1 |
| S. felis | 1 |
| S. gallinarum | 1 |
| S. haemolyticus | 3 |
| S. lentus | 2 |
| S. hominis | 1 |
| S. hyicus | 5 |
| S. intermedius | 3 |
| S. kloosii | 1 |
| S. lugdunensis | 2 |
| S. muscae | 1 |
| S. saprophyticus | 3 |
| S. schleiferi | 1 |
| S. sciuri | 3 |
| S. simulans | 1 |
| S. simulans | 2 |
| S. unknown | 1 |
| S. vitulus | 1 |
| S. warneri | 4 |
| S. xylosus | 4 |
| S. xylosus | 1 |
| Stenotrophomonas | 1 |
| maltophilia | |
| Stomatococcus | 1 |
| mucilaginosus | |
| Streptococcus equi | 1 |
| S. pneumoniae | 1 |
| S. pyogenes | 2 |
| S. salvarius | 1 |
| Yersinia enterolytica | 1 |

TABLE 4
Results for Campylobacter strains tested

| | # strains | % positive for | % positive for |
|---|---|---|---|
| Species | tested | C. jejuni | C. coli |
| C. jejuni | 115 | 100 | 0 |
| C. coli | 32 | 0 | 100 |
| C. hyoilei¹ | 6 | 0 | 100 |
| C. fetus ss. fetus | 3 | 0 | 0 |
| C. fetus ss. venerealis | 3 | 0 | 0 |
| C. hyointestinalis | 5 | 0 | 0 |
| C. lari | 25 | 0 | 0 |
| C. upsaliensis | 1 | 0 | 0 |
| Arcobacter butzleri | 8 | 0 | 0 |

¹Six strains of C. hyoilei tested positive for the C. coli primer set. These results are as expected because C. hyoilei is considered to be a junior synonym to C. coli (18).

Each primer set within this assay has demonstrated 100% inclusivity for its respective targets and 100% exclusivity for all non-target organisms tested.

The sensitivity of the multiplex PCR is between one and 10 target bacteria for each of the primer sets.

These PCR results demonstrate an improvement over existing detection methods for Campylobacter (U.S. Pat. No. 6,080,547). This patent discloses the detection of four Campylobacter species with PCR, C. coli, C. jejuni, C. lari, and C. upsaliensis, but requires a restriction digest step in order to distinguish the individual species. The present invention independently identifies C. coli and C. jejuni, the only Campylobacter species that are pathogenic to humans.

An additional method for detection of Campylobacter strains in disclosed in U.S. Pat. No. 6,066,461. This patent discloses the use of Strand Displacement Amplification (SDA), not PCR, and requires radioactive isotope for probe-based detection of the SDA product. The sensitivity of this assay is 100 cells, whereas the sensitivity of the present invention ranges between one and 10 cells.

### Example 2

The multiplex PCR for C. coli and C. jejuni was also performed on the Automated BAX® system, which uses melting curve detection. A positive reaction for C. coli resulted in the presence of a melting curve peak at 82.5° C. (FIG. 3).

FIG. 4 shows the melting curve results for a sample that contained both C. coli and C. jejune. The C. jejuni PCR product melts at 80.5° C., which is clearly discernable from the C. coli melting curve peak at 82.5° C. The multiplex PCR was further expanded by the incorporation of an Internal Positive Control (INPC). Reagents for the INPC (target DNA and primers) were added to the Campylobacter multiplex reaction containing primer sets PS1 and PS2.

FIG. 5 shows the melting curve results for a C. coli positive sample. The INPC has a melting curve peak at 78° C., whereas the C. coli melting curve peak remains at 82.5° C. The incorporation of the INPC provides the user with a one-tube test that will indicate whether C. coli and/or C. jejuni are present, and will also indicate that the test worked properly (INPC result) when neither C. coli nor C. jejuni were present.

What is claimed is:

1. A method for detecting a pathogenic Campylobacter species in a sample, the method comprising:
(a) preparing the sample for PCR amplification
(b) performing PCR amplification of the sample using a combination of PS1 (SEQ ID NOs: 1 and 2) and PS2 (SEQ ID NOs: 3 and 4) primers; and
(c) examining the PCR amplification result,
whereby a positive amplification indicates the presence of a pathogenic Campylobacter species.

2. The method of claim 1, wherein step (a) comprises at least one of the following processes: (1) bacterial enrichment, (2) separation of bacterial cells from the sample, (3) cell lysis, and (4) total DNA extraction.

3. The method of claim 1, wherein the pathogenic Campylobacter species is Campylobacter jejuni or Campylobacter coli.

4. The method of claim 1, wherein the sample comprises a food or a water sample.

5. A method for detecting Campylobacter coli in a sample, the method comprising:
(a) preparing the sample for PCR amplification
(b) performing PCR amplification of the sample using PS1 primers (SEQ ID NOs: 1 and 2); and
(c) examining the PCR amplification result,
whereby a positive amplification indicates the presence of a pathogenic Campylobacter coli in the sample.

6. The method of claim 5, wherein step (a) comprises at least one of the following processes: (1) bacterial enrichment, (2) separation of bacterial cells from the sample, (3) cell lysis, and (4) total DNA extraction.

7. A method for detecting Campylobacter jejuni in a sample, the method comprising:
(a) preparing the sample for PCR amplification
(b) performing PCR amplification of the sample using PS2 (SEQ ID NOs: 3 and 4) primers; and
(c) examining the PCR amplification result,
whereby a positive amplification indicates the presence of Campylobacter jejuni in the sample.

8. The method of claim 7, wherein step (a) comprises at least one of the following processes: (1) bacterial enrichment, (2) separation of bacterial cells from the sample, (3) cell lysis, and (4) total DNA extraction.

9. An isolated polynucleotide for the specific detection of Campylobacter coli, consisting essentially of the nucleic acid sequence of SEQ ID NO: 1 or SEQ ID NO: 2.

10. An isolated polynucleotide for the specific detection of Campylobacter jejuni, consisting essentially of the nucleic acid sequence of SEQ ID NO: 3 or SEQ ID NO: 4.

11. The method according to claim 1 wherein the sample comprises a selectively enriched food matrix.

12. A kit for the detection of a pathogenic Campylobacter species selected from the group consisting of Campylobacter jejuni and Campylobacter coli in a sample, the kit comprising:
(a) at least one pair of PCR primers selected from the group consisting of PS1 (SEQ ID NOs: 1 and 2) and PS2 (SEQ ID NOs: 3 and 4); and
(b) a mixture of suitable PCR reagents comprising a thermostable DNA polymerase.

13. The method according to claim 1 wherein the mixture of suitable PCR reagents is provided in a tablet.
